(19) United States
(12) Patent Application Publication
(10) Pub. No.: US 2020/0324078 A1
(43) Pub. Date: Oct. 15, 2020

(54) INFECTION RESISTANT CATHETER SYSTEM

(71) Applicants: Cecil Fred MOTLEY, Rolling Hills, CA (US); Randall W. MAXEY, Rolling Hills Est, CA (US)

(72) Inventors: Cecil Fred MOTLEY, Rolling Hills, CA (US); Randall W. MAXEY, Rolling Hills Est, CA (US)

(21) Appl. No.: 16/381,431

(22) Filed: Apr. 11, 2019

(51) Int. Cl. A61M 25/00; A61L 2/00; A61L 2/10; A61M 39/16

(52) U.S. Cl. CPC A61M 25/0017; A61L 2/0047; A61L 2/10; A61M 25/0097; A61L 2202/24; A61M 2039/167; A61M 39/16

(57) ABSTRACT

This invention is for a catheter apparatus that greatly reduces microbial infections resulting from catheterization of dialysis, semi-mobile, or hospitalized patients. The apparatus applies light in the ultraviolet and near ultraviolet band to multiple catheter lumens for both detection and inactivation of biofilm microorganisms. It uses real-time automated techniques for the selection of wavelength, power level and exposure time regimes that are used for irradiating the biofilm in vivo. Artificial intelligence is incorporated to adjust the UV irradiation regimes to maximize the microorganism inactivation efficacy while minimizing the destruction of keratinocytes. The biofilm inactivation efficacy of this infection resistant catheter apparatus is at least 99%. The apparatus allows for minimal deviation from conventional catheter insertion procedures and can remain in vivo for long periods of time without the risk of microorganism contamination.

### PUBLICATION CLASSIFICATION

| | |
|---|---|
| A61L 2/0047 | (January 2013) |
| A61M 25/00 | (January 2013) |
| A61M 25/0026 | (January 2013) |
| A61M 2025/0004 | (January 2013) |
| A61M 2025/0019 | (January 2013) |

### REFERENCES CITED

U.S. PATENT DOCUMENTS

| | | |
|---|---|---|
| 4,698,058 A | Oct. 6, 1987 | Greenfield et al |
| 5,240,675 A | Aug. 31, 1993 | Wilk et al |
| 5,260,020 A | Nov. 9, 1993 | Wilk et al |
| 9,550,005 B2 | Jan. 24, 2017 | Lin et al. |
| 9,492,574 B2 | Nov. 15, 2016 | Rasooly et al. |
| 8,946,653 B2 | Feb. 3, 2015 | Victor et al. |
| 9,320,880 B2 | Apr. 26, 2016 | Levenson et al. |
| 9,295,742 B2 | Mar. 29, 2016 | Rasooly et al. |
| 9,259,513 B2 | Feb. 16, 2016 | Bedwell et al. |
| 8,911,424 B2 | Dec. 16, 2014 | Weadock et al. |
| 8,933,416 B2 | Jan. 13, 2015 | Arcand et al. |
| 6,461,569 B1 | Oct. 8, 2002 | Boudreaux |
| 5,334,171 A | Aug. 2, 1994 | Kaldany |
| 20160082138 A1 | Mar. 24, 2016 | Kermode et al. |
| 20150352348 A1 | Dec. 10, 2015 | Murphy-Chutorian et al |
| 20150165185 A1 | Jan. 18, 2015 | Cohen et al. |
| 20150126976 A1 | May 7, 2015 | Tang et al. |
| 20140334974 A1 | Nov. 13, 2014 | Rasooly et al. |
| 20130060188 A1 | Mar. 7, 2013 | Bedwell et al. |
| 20120321509 A1 | Dec. 20, 2012 | Bak |
| 20110160644 A1 | Jun 30, 2011 | Dacey, Jr. et al. |
| 20110213339 A1 | Sep. 1, 2011 | Bak |
| 20090257910 A1 | Oct. 15, 2009 | Segal |

### OTHER PUBICATIONS

⁽¹⁾ “The Use of UV Light to Reduce Infections Associated with Central Venous, Arterial, and Urinary Catheters”, Motley et al, CMS Publication, Jul. 16, 2015.

⁽²⁾ “UV Inactivation of Pathogenic and Indicator”, Chang et al, APPLIED AND ENVIRONMENTAL MICROBIOLOGY, June 1985, Vol. 49, No. 6, p. 1361-1365.

### BACKGROUND OF THE INVENTION

### 1. Field of the Invention

This invention meets the need to eliminate infections caused by central venous, vascular, urinary, and indwelling catheters required for intravascular access management of dialysis, semi-mobile, and hospitalized patients. The method and apparatus used to implement infection resistant catheters described in this disclosure is based on the application of concentric multi-lumen catheters that carry ultraviolet (UV) light for both detection and inactivation of bacteria, viruses, and other microorganisms, also known as biofilms, that accumulate on the external surface of the catheter. The disclosed catheter apparatus inactivates this biofilm and is henceforth referred to as the Infection Resistant Catheter (IRC) or Motley-Maxey Catheter (MMC).

The MMC inactivates microorganism contamination by first sensing the presence of said microorganisms using fluorescing techniques and then irradiating the catheter lumens with UV light in the C-band (UVC) in vivo (while inserted in the patient). The process continues automatically as long as the catheter remains in the patient. The efficacy of the present invention is measured by the amount of cellular inactivation produced by exposing the bacteria, virus, or other microorganism to UVC radiation without the loss of keratinocytes. The intensity level, irradiation frequency, time of exposure, and dose regime determines the degree of inactivation of the particular microorganism. In the present invention, these parameters are automatically determined during real-time operation of the MMC. The process is based on the application of artificial intelligence (AI) technology to optimize the irradiation regime to minimize the loss of keratinocytes while maximizing the microorganism inactivation efficacy.

The biofilms on the outer surface of the catheters become antibiotic resistant over time thus rendering current treatment of biofilm ineffective. The biofilm also becomes resistant to a particular UV light irradiation regime over time. To prevent the microorganisms in the biofilm from adapting to the UV irradiation regime, the AI algorithms automatically adjust the regime parameters to re-establish effective inactivation of the microorganisms.

### 2. Background Art

The management of critically ill patients is virtually impossible without intravascular or indwelling access devices. These devices include central venous, indwelling, urinary, and vascular catheters, which are long thin flexible tubes used to give medicines, fluids, nutrients, or blood products over a long period of time, usually several weeks or more. For dialysis patients, a central venous catheter (CVC) is often inserted in the arm, chest or neck through the skin into a large vein. The catheter is threaded through this vein until it reaches a large vein near the heart. CVCs are widely used in the United States, and it is estimated that physicians insert more than 5 million CVCs every year.

Although the CVC has been shown to have numerous medical benefits, one of the more common risks associated with their use is the occurrence of catheter-related infections. Urinary, intravascular, and indwelling catheters are also known to have the potential to allow access of bacteria or other microorganisms into the bloodstream or surrounding tissue. The CVC, because of its placement and long term use, is the most susceptible to becoming contaminated in a manner which can cause serious infection by microorganisms such as Staphylococcus aureus and Staphylococcus epidermidis sepsis. The CVC is now the most frequent source of nosocomial bloodstream infection, and it has been estimated that 250,000-500,000 episodes occur in the United States annually, with an estimated 10% mortality and marginal cost to the health care system of $45,000 per episode.

Current antimicrobial technology includes catheters coated or impregnated with special materials and usage procedures intended to reduce bacterial and viral contamination. Other approaches to reducing infection occurrences include heated wires running along the catheter lumens, channels that allow the use of clean out wires, acoustical vibrating channels along the lumen walls, and catheter antibiotic gels and creams. Additionally, catheter manufacturing and sterilization techniques include exposing the catheters to UV light before and during the packaging process. UV light is also currently being used to sterilize catheters both in vitro and in vivo.

For example, U.S. Pat. No. 4,698,058 A to Greenfield et al. discloses the use of vibrations that are conveyed to the proximal orifices of an indwelling catheter to disintegrate accumulated clogging deposits, large suspended particles and contaminating bacteria, viruses, fungi, etc. Vibrations are conveyed to the orifices by (1) a solid fiber embedded in the catheter walls or (2) by a liquid in an auxiliary lumen.

U.S. Pat. Nos. 5,240,675 A and 5,260,020 A to Wilk et al. discloses a method for cleaning a flexible endoscope, using an elongate cleaning member embedded optical fiber, an electrical conductor, and/or a heat conductor inserted into a biopsy channel of the endoscope. Sterilizing radiation of a predetermined wavelength is transmitted along the optical fiber from a proximal end of the elongate cleaning member towards a distal end thereof. The radiation is dispersed to at least partially sterilize the biopsy channel.

U.S. Pat. No. 9,550,005 B2 to Lin et al. discloses a method to sterilize catheters with single or multiple lumens by inserting a fiber optic cable to deliver UV energy. The technique applies maximum energy inside the lumen with much attenuated energy reaching the external catheter surface where biofilm infections are formed.

U.S. Pat. No. 9,492,574 B2 to Rasooly et al. discloses a method and apparatus involving an elliptical-shaped removable housing that is wrapped around the catheter connector to the lumens. This apparatus delivers UV light to the fluid passing through the proximal end of the catheter lumens without making direct contact with the fluid. Its primary purpose is to keep free from bacteria the connector end of the catheter and fluid passing through this connection.

U.S. Pat. No. 8,946,653 B2 to Victor et al. discloses a UVC based device for sterilization of the catheter insertion site and associated catheter during insertion. This method provides a prophylactic measure that replaces the isopropyl alcohol cleaning and sterilization procedures normally applied by health care personnel.

U.S. Pat. No. 9,320,880 B2 to Levenson et al. discloses a device that exposes fluid flowing through a catheter system prior to entering the patient. This method ensures decontamination of any material delivered through the IV or indwelling catheter.

U.S. Pat. No. 9,295,742 B2 and 2014/0334974 A1 to Rasooly et al. discloses a system for disinfecting fluid associated with peritoneal dialysis. Primary and secondary UV light sources are used based on a detection system located at the hub of the catheter.

U.S. Pat. No. 9,259,513 B2 and 2013/0060188 A1 to Bedwell et al. discloses a catheter system that uses UV light to activate oxidizing agents (Reactive Oxygen Species) on the outer surface of the catheter. This technique provides a photocatalytic disinfection without removing the catheter.

U.S. Pat. No. 8,911,424 B2 to Weadock et al. discloses a collar for urinary catheters that contains antimicrobial hydrogel agents that are released in the urinary tract to prevent infections.

U.S. Pat. No. 8,933,416 B2 to Arcand et al. discloses a UV based pre and post insertion device for catheter devices. A UV based sheath insertion device is used to pre-sterilize the catheter to prevent microbial material from being inserted into the catheter site. The device is also used for post insertion to re sterilize the catheter assembly.

U.S. Pat. No. 6,461,569 B1 to Boudreaux discloses a removal sterilization system that delivers UV light within the catheter lumen wall using a fiber optics cable that emits light normal to its outer surface. This device is inserted when needed and emits continuous light when applied.

U.S. Pat. No. 20160082138 A1 and 20150352348 A1 to Kermode et al. discloses a disinfection unit that is attached to the tubing connecting the dialate container used in peritoneal dialysis to the catheter inserted into the patient. The device uses UV light to irradiate the solution and the tubal connection to the indwelling catheter.

U.S. Pat. No. 20150165185 A1 to Cohen et al. discloses a method to provide catheters and catheter connections UV sterilization of fluid as it flows through catheters and associated connections.

U.S. Pat. No. 20150126976 A1 to Tang et al. discloses a flexible drainage catheter that includes an internal UV irradiating member that is used to sterilize the internal surface. The irradiating device is a fiber optics wire that irradiates through its surface wall.

U.S. Pat. No. 20120321509 A1 and 20110213339 A1 to Bak discloses a coupling device that delivers UV light and other forms of radiation to catheter lumens. It is useable on existing and specially designed catheters.

U.S. Pat. No. 20110160644 A1 to Dacey, Jr. et al discloses an implantable device that when attached to a catheter tube delivers UV energy that internally releases absorbing agents. This process is computer controlled and consists of several irradiation protocols.

Although the aforementioned inventions use various techniques incorporating UV light, they fail to provide a system that eliminates the bacteria and virus associated with catheter devices while minimizing the destructive nature of UV light on skin tissue. This invention uses advance AI controlled irradiation regimes to optimize the efficacy while minimizing the destruction of keratinocytes.

### SUMMARY OF THE INVENTION

This disclosure is for a catheter system that provides a significant improvement in the prevention of catheter related infections due to long term use of central venous, intravenous, urinary, and indwelling catheters. The invention applies light in the UVC band to inactivate the biofilm that accumulates on the surfaces of the catheter in vivo. An associated computer executing Artificial Intelligence (AI) algorithms control the UVC light source and detection system that are coupled to the catheter through fiber optic cables. The AI design automatically detects the types of microorganisms present on the surface of the catheter and creates the optimum UV based irradiation protocol that inactivates the bacteria, while protecting keratinocytes from damage or destruction. The AI created detection and irradiation regimes also defend against bacteria adaptability to the UV inactivation sequences.

### BRIEF DESCRIPTION OF THE DRAWINGS

The invention is illustrated by way of example and not by way of limitation in the figures of the accompanying drawings in which like references indicate similar elements. It should be noted that references to “an” or “one” embodiment in this disclosure are not necessarily to the same embodiment, and such references mean at least one.

FIG. 1 illustrates the pathways that are subject to microorganism contamination that can lead to infection.

FIG. 2 illustrates one embodiment of the invention having all the elements of the lumen structure associated with a single injectate infection resistant catheter.

FIG. 3 illustrates one embodiment of the invention having all the elements of the multiple lumen structure associated with a multi injectate or injectate/drain infection resistant catheter.

FIG. 4 illustrates the fluid filled jacket used to irradiate or receive light to and from the full length of the surface of the infection resistant catheter.

FIG. 5 illustrates a block diagram of the infection resistant catheter light source and control unit.

FIG. 6 illustrates the Deep Learning Neural Net employed by the AI algorithms associated with this invention.

FIG. 7 illustrates one embodiment of this invention having all the elements of the multi-lumen infection resistant catheter and its associated AI control unit.

### DETAILED DESCRIPTION AND CLAIMS

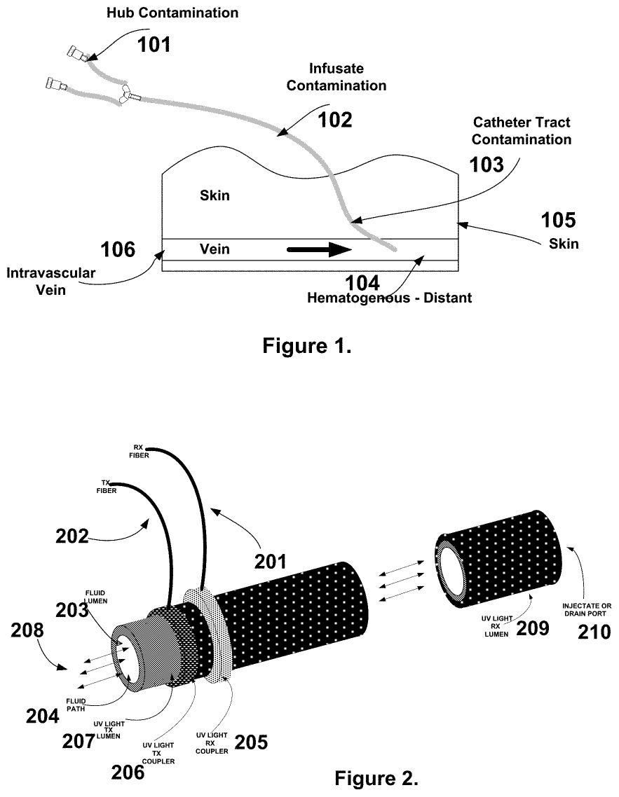

There are four distinct pathways that lead to catheter-related infection (FIG. 1.0).
1. First, colonization of the outer surface may start by the migration of skin resident microorganisms from the insertion site, and microbial cells may progressively move through the transcutaneous part of the dermal tunnel surrounding the catheter 103.
2. Second, colonization of the internal surface may occur by colonization of the hub and intraluminal surface of the catheter during utilization, and frequent opening of the hub is now viewed as an important source of microbial colonization 101.
3. Hematogenous seeding of the catheter during bloodstream infection of any origin represents a third pathway 104, and
4. Fourth, contamination of the fluids or drugs intravenously administered is sometimes responsible for outbreaks 102.

The apparatus described in this enclosure is resistant to all four contamination sources. It uses UVC light to damage the genetic material in the nucleus of the microorganism cell⁽¹⁾. UVC light in the range of 180 to 270 nm is strongly absorbed by the nucleic acids of an organism. The light induced damage to the DNA and RNA of an organism often results from the dimerization of pyrimidine molecules. In particular, thymine (which is only found in DNA) produces cyclobutane pyrimidine dimers. When these molecules are dimerized, it becomes very difficult for the nucleic acids to replicate and if replication does occur it often produces a defect which prevents the microorganisms from being viable.

Previous studies have shown that 254 nm is near optimum for germicidal effects on microorganisms. In 1878, Arthur Downes and Thomas P. Blunt published a paper describing the sterilization of bacteria exposed to ultraviolet UV light^{[2]} in the 250 nm to 280 nm range. At these wavelengths, UV light is mutagenic to bacteria, viruses and other microorganisms. This process is similar to the effect of UV wavelengths that produce sunburns in humans. Microorganisms have less protection from UV light and cannot survive prolonged exposure to it.

The embodiment of this disclosure is the multi-lumen mechanical structure, the fiber optics AI based UV light irradiation delivery and detection system, and the control algorithms that maximize the infection resistance of the catheter while minimizing the UV destruction of keratinocytes by exploiting the mutagenic behavior of UV light on microorganisms.

With reference to FIG. 2, a single fluid flow example of the disclosed apparatus operates as follows: There are three catheter lumens in this example, a UV light transmitter jacket 207, a UV light receiver jacket 209, and a fluid transfer lumen 203. The UV light transmit lumen 207 irradiates the entire length of the catheter with 240 to 280 nm light to inactivate microorganisms at the intraluminal surface 209, hub, hematogenous distant tip and in the fluid 208 flowing through the catheter. Alternately, this same UV light transmit lumen 209 irradiates the entire length of the apparatus with wavelengths in the UVC band for the purpose of fluorescing microorganisms that may be present at any of the catheter sites subject to infection. The light from the fluoresced microorganisms is detected by sensors linked to the received light catheter lumen 209. This alternating process continues until no fluoresced microorganisms are detected. The alternating detection and irradiation cycle is controlled by AI algorithms running on a processor located in an external control unit attached to the catheter by fiber optic links 201, 202. The specific wavelength, power level, and “on-time cycle” are also controlled by AI algorithms running on the external control unit. The continuous changing of irradiation wavelength controlled by the AI algorithms, suppresses the ability for adaptation to inactivation by the microorganisms. The light sent and received by the transmit and receive lumens is coupled to the sources and sensors using optical couplers 205, 206 placed at the catheter entry sites. When the presence of microorganisms is not detected, the irradiation algorithm applies maintenance doses only of UV which also inactivate microorganisms that do not fluoresce but are still harmful to the patient. This single fluid flow example of the disclosed apparatus forms the baseline operational structure for other examples of the infection resistant catheter apparatus.

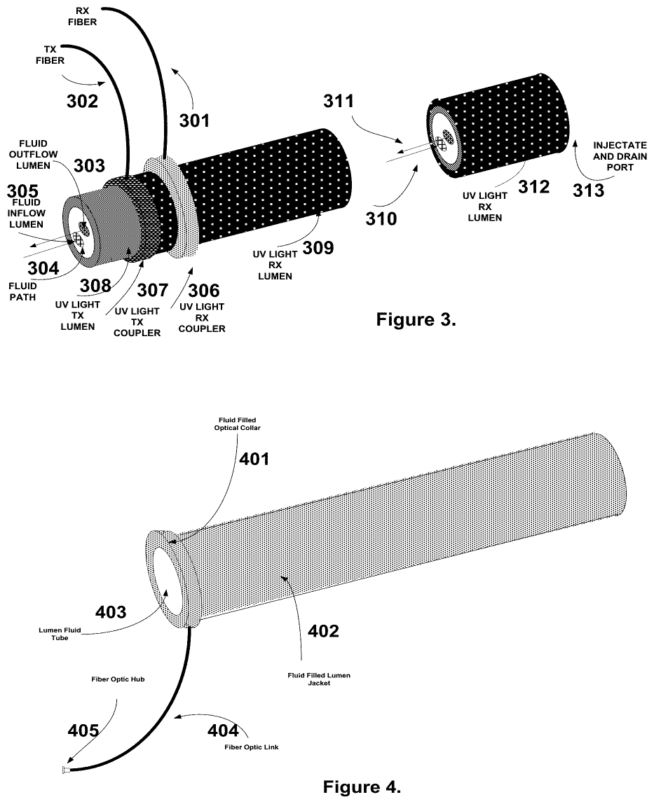

With reference to FIG. 3, a multi-flow multi-lumen example of the disclosed apparatus operates as follows: There are five catheter lumens in this example, a UV light transmit 308, UV light receive 312, and two fluid transfer lumens (304, 303) respectively. The UV light transmit lumen 308 irradiates the entire length of the catheter with 240 to 280 nm light to inactivate microorganisms at the intraluminal surface 312, hub, hematogenous distant tip 313, and in the fluid (303, 304) flowing through the catheter. This example of the apparatus is identical to the single flow version and operates the same with the exception of having two fluid flow lumens. The two lumens are generally used in central venous catheter applications to carry blood to and from the right atrium heart chamber.

With reference to FIG. 4, a fluid filled UV light transport lumen example of the disclosed apparatus follows: Each catheter type has a UV light transmit lumen and a UV light receive lumen. These lumens consist of distilled water filled jackets 402 due to the extreme attenuation of UV light in the 240 to 280 nm wavelengths encountered when the jackets are solely implemented with polyethylene plastic (a plastic having the least amount of UV attenuation). Instead, a very thin polyethylene fluid filled jacket is used. An optical coupler 401, also fluid filled, connects a fiber optics cable 404 that delivers the light to the coupler from an LED source in a control unit through an optical hub 405. The single or multi-flow fluid transfer lumens are inserted in the light carrying jackets 403 to complete the catheter implementation.

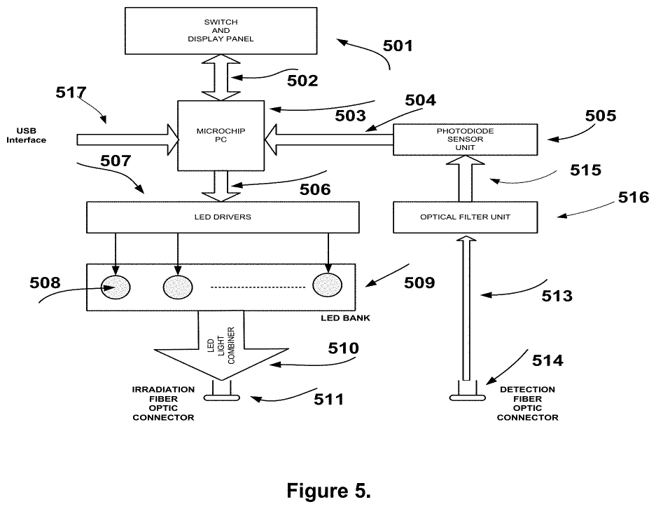

With reference to FIG. 5, a functional block diagram of the control unit of the disclosed apparatus is provided. The purpose of the control unit is to execute an AI directed protocol that selects the “ON-OFF-SWITCHING”, “PULSE DURATION”, “IRRADIATION WAVELENGTH SELECTION”, “FLORECENSE WAVELENGTH SELECTION, and “POWER LEVEL” required to inactivate bacteria or virus microorganisms present on the catheter external surface. A deep learning neural net AI algorithm implemented in software running on an embedded processor 503 within the control unit executes this operation.

The components of the control unit include:
a source of UV light when connected to the lumen fiber optic hub (510);
a source for providing the “ON TIME” for UV irradiation (508);
a source for providing irradiation wavelength (508,509);
a source for providing the irradiation power level (507);
a sensor for detecting presence of fluoresced microorganisms (505);
a embedded micro-processor 503 for executing artificial intelligence software that automatically determines the power level, irradiation wavelength, and irradiation “ON TIME” that maximizes the efficiency of deactivating the biofilm microorganisms while minimizing the destruction of keratinocytes associated with the insertion site;
an interface to an external device via USB (cell phone, laptop computer, online connected device (517);
an optical filter unit for separating irradiation wavelengths from fluoresced wavelengths (516);
an internal interface bus (504);
a fiber optics hub connecting the irradiating sources to catheter fiber optics cables (511, 514);
a manual operating switch and associated display panel (501).

The control unit has an embedded internal microchip PC 503 which executes the algorithms and protocols associated with the MMC system. The “ON TIME” algorithm running on the embedded PC signals 506 the LED drivers 507 “turn-on” times for the UV light LED array 509. The specific wavelength is also controlled by the embedded PC such that individual or concurrent LEDs 508 are “turned on” based on the type of microorganism that is to be inactivated or detected.

The operational sequence starts with the control unit in the detection mode whereby the LED array outputs a 300 to 780 nm light to the catheter fiber optic transmit hub 511. This near UV light energy is used to florescenced microorganisms that may be present along the outer walls, insertion site, or inner fluid of the catheter. The specific type of biofilm is determined by the wavelength of the fluoresced microorganisms which is determined by the optical filter bank 516 and sent 515 to a photodiode detector sensor 505 in the control unit. The level of the detected fluoresce is transferred 504 to the microchip PC 503 where the AI algorithm determines what wavelength, “ON TIME”, and power level should be used to inactivate the biofilm. The microchip PC 503 then enables the appropriate LED drivers 507 to deliver UV light in the 180 to 270 nm band at the fiber optics transmit hub 511. The switch and display panel 501 allows the operator to power on the unit, select the inactivation protocol, select the automatic mode algorithm, and select the reporting action through local alarm, through a smartphone via USB interface 517, or an external PC. The smartphone interface enables contamination status to be reported to healthcare personnel remotely.

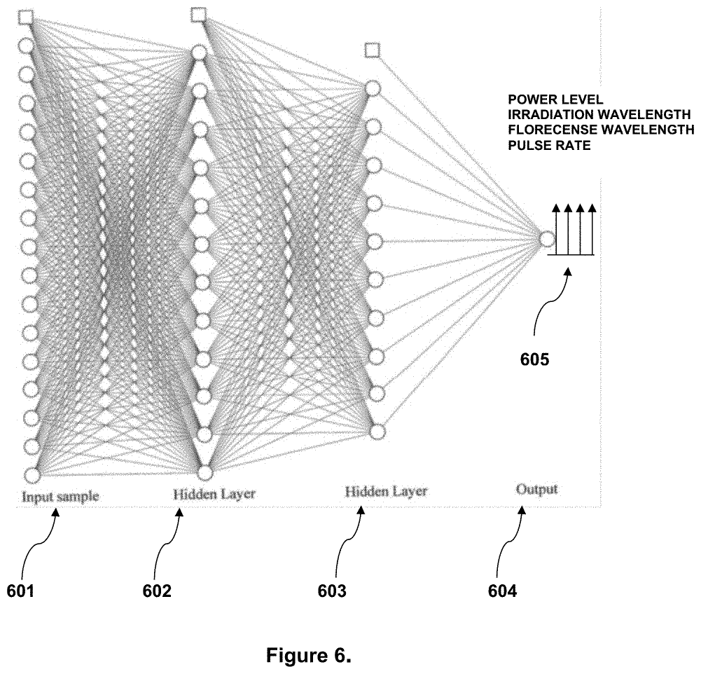

FIG. 6 illustrates the layers of the deep learning neural net that identifies the microorganism patterns present on the catheter surface. This identification is based on sixteen samples 601 of surface biofilm fluoresced by sixteen different wavelengths. Based on the florescence pattern detected, the deep learning neural net 602, 603, 604 creates an irradiation regime 605 that completely inactivates the biofilm microorganisms while causing minimum damage to keratinocytes. A bank of eight different irradiation sources is controlled by the deep learning neural net.

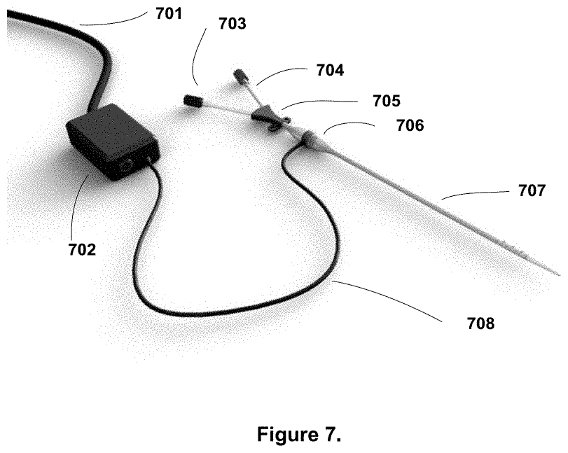

FIG. 7 illustrates one embodiment of this invention as a complete dual lumen catheter system. The catheter is connected to the control unit 702 with an attached fiber optics cable 708, thus alleviating the possibility of electrical shock to the patient. The control unit is reusable with any MMC type catheter while the actual catheter unit 703, 704, 705, 706, and 707 is disposable. The disposable portion of the catheter has the usual elements of a traditional dual lumen catheter. A power cable 701 is included to recharge batteries during mobile use or connect to A/C source during long term use.

1. An apparatus comprising:
An infection resistant catheter system that provides UVA and UVB wavelength light to fluoresce biofilm bacteria on the exterior and interior walls of an in-vivo catheter to produce a spectral pattern of the biofilm bacteria and UVC wavelength light irradiation to inactivate the biofilm bacteria while minimizing keratinocyte destruction. Further, two concentric liquid jackets deliver the UVC irradiation and fluoresced UVA and UVB light to all internal, external walls and hub elements of the catheter. Further, a Deep Learning Neural Network and associated bacteria spectral pattern training data automatically manage the biofilm spectral pattern detection, wavelength selection, power level and irradiation source protocol.
The apparatus components include:
a plurality of lumens flexible tube structure surrounded by two liquid filled concentric jackets;
a UVA, UVB, and UVC band optical transmit liquid coupler;
a UVA, UVB, and UVC band optical receive liquid coupler;
a fiber optic transmit cable;
a fiber optic receive cable;
a plurality of UVA, UVB, and UVC band light sources;
a plurality of UVA, UVB, and UVC band optical filters;
a plurality of UVA, UVB, and UVC band photo sensors;
a high speed graphic processing unit
a eight layer Deep Learning Neural Network with a plurality of input and output nodes;
a Deep Learning Neural Network training data set for 63 fluoresced bacteria spectral patterns;
a bacteria inactivation irradiation protocol algorithm;
a bacteria type and state detection Deep Learning Neural Network directed protocol algorithm;
a irradiation protocol control algorithm;
a battery power source;
a rechargeable power source unit;
a set of multi-lumen hubs and
a injectate or drain port distal tip.

2. The apparatus of claim 1 wherein a plurality of fluid carrying lumens are used as a dialysis central venous catheter (CVC).

3. The apparatus of claim 1 wherein a single fluid carrying lumen is used as an intravascular catheter (IVC).

4. The apparatus of claim 1 wherein a single fluid carrying lumen is used as a urinary catheter (UC).

5. The apparatus of claim 1 wherein a plurality of fluid and gas carrying lumens are used as an inflatable tip urinary catheter (ITUC).

6. The apparatus of claim 1 wherein a plurality of fluid and gas carrying lumens are used as a pulmonary indwelling catheter (PIDC).

7. The apparatus of claim 1 wherein a Deep Learning Neural Network directed irradiation wavelength is automatically adjusted to suppress microorganism adaptation to the UVC inactivation light.

8. The apparatus of claim 1 wherein a Deep Learning Neural Network directed detection algorithm is used to signal the presence of fluoresced microorganisms on any of the catheter walls.

9. The apparatus of claim 1 wherein a plurality of fluid carrying lumens are used as an indwelling fluid delivery or drain catheter (IDC).

10. (canceled)

11. The apparatus of claim 1 wherein two liquid jackets are used to carry UVA, UVB and UVC band light to all lumens and associated hubs.

12. The apparatus of claim 1 wherein a liquid jacket is used to carry UVA and UVB band light from fluoresced microorganisms from all associated catheter internal and exterior walls.

13. The apparatus of claim 1 wherein the irradiation “ON TIME” is automatically adjusted by a Deep Learning Neural Network to minimize keratinocyte destruction while insuring up to 99.9% inactivation of the catheter internal and exterior walls biofilm microorganisms.

14. The apparatus of claim 1 wherein the irradiation “POWER LEVEL” is automatically adjusted by a Deep Learning Neural Network to minimize keratinocyte destruction while insuring up to 99.9% inactivation of the catheter internal and exterior walls biofilm microorganisms.

15. The apparatus of claim 1 wherein the irradiation “WAVELENGTH” is automatically adjusted by a Deep Learning Neural Network to prevent viability adaptation of the target bacteria or virus on the catheter internal and exterior walls biofilm microorganisms.

16. The apparatus of claim 1 wherein the irradiation regime (i.e. wavelength, “ON TIME”, “POWER LEVEL”, detection protocol, and pulse rate) is controlled by a Deep Learning Neural Network executed on an embedded graphic processor in the catheter control unit.

17. The apparatus of claim 1 wherein Deep Learning Neural Net algorithms are executed on the embedded graphic processor.
